(19) United States
(12) Patent Application Publication
(10) Pub. No.: US 2021/0052846 A1
(43) Pub. Date: Feb. 25, 2021

(54) PRESENTATION CONTROL DEVICE, NON-TRANSITORY COMPUTER READABLE MEDIUM, AND RESPIRATORY CYCLE PRESENTATION DEVICE

(71) Applicant: FUJI XEROX CO., LTD., Tokyo (JP)

(72) Inventors: Toshihiko SUZUKI, Kanagawa (JP); Taijyu GAN, Kanagawa (JP)

(73) Assignee: FUJI XEROX CO., LTD., Tokyo (JP)

(21) Appl. No.: 16/811,307

(22) Filed: Mar. 6, 2020

(30) Foreign Application Priority Data
Aug. 23, 2019 (JP) 2019-153052

(51) Int. Cl. A61M 21/02

(52) U.S. Cl. CPC A61M 21/02; A61M 2021/0016

(57) ABSTRACT

A presentation control device includes a processor. The processor is configured to perform first control for presenting a first respiratory cycle longer than a natural respiratory cycle for a respiratory cycle presentation device in a case of preferentially increasing a degree of relaxation, the respiratory cycle presentation device being a device that presents a respiratory cycle, and perform second control for presenting a second respiratory cycle for the respiratory cycle presentation device in a case of preferentially decreasing a degree of physical-mental activity, the second respiratory cycle being a cycle for which a difference between the second respiratory cycle and the natural respiratory cycle is smaller than a difference between the first respiratory cycle and the natural respiratory cycle.

### CROSS-REFERENCE TO RELATED APPLICATIONS

This application is based on and claims priority under 35 USC 119 from Japanese Patent Application No. 2019-153052 filed Aug. 23, 2019.

### BACKGROUND

### (i) Technical Field

The present disclosure relates to a presentation control device, a non-transitory computer readable medium, and a respiratory cycle presentation device.

### (ii) Related Art

Japanese Unexamined Patent Application Publication No. 2010-104457 discloses a refreshment guiding system that includes: a relaxation guiding means for creating an environment for making a parasympathetic nerve system dominant in an autonomic nerve function of a user; a breathing guiding means for giving a target value only for a breathing-out period among a breathing-in period and the breathing-out period of the user to make the breathing-out period longer, the breathing guiding means including a breathing information presentation means for presenting to the user changes in a state of breathing of the user detected by a respiration sensor as breathing information; and a guiding control means for causing the breathing guiding means to start operating at a time point when it is inferred that the parasympathetic nerve system of the user becomes dominant as a result of use of the relaxation guiding means.

Japanese Unexamined Patent Application Publication No. 2002-336358 discloses a breathing regulation method using a breathing regulation device for regulating breathing of a person, in which a period for which breathing of the person is regulated is set to a period with which a degree of awakening of the person does not decrease.

### SUMMARY

In a control device that performs control to present a respiratory cycle longer than a natural respiratory cycle in a case of preferentially increasing the degree of relaxation, when the above-described control is performed also in a case of preferentially decreasing the degree of physical-mental activity, the degree of physical-mental activity might not decrease.

Aspects of non-limiting embodiments of the present disclosure relate to a configuration with which the degree of physical-mental activity is decreased unlike with a configuration in which control is performed to present a respiratory cycle longer than a natural respiratory cycle also in the case of preferentially decreasing the degree of physical-mental activity.

Aspects of certain non-limiting embodiments of the present disclosure overcome the above disadvantages and/or other disadvantages not described above. However, aspects of the non-limiting embodiments are not required to overcome the disadvantages described above, and aspects of the non-limiting embodiments of the present disclosure may not overcome any of the disadvantages described above.

According to an aspect of the present disclosure, there is provided a presentation control device including a processor. The processor is configured to perform first control for presenting a first respiratory cycle longer than a natural respiratory cycle for a respiratory cycle presentation device in a case of preferentially increasing a degree of relaxation, the respiratory cycle presentation device being a device that presents a respiratory cycle, and perform second control for presenting a second respiratory cycle for the respiratory cycle presentation device in a case of preferentially decreasing a degree of physical-mental activity, the second respiratory cycle being a cycle for which a difference between the second respiratory cycle and the natural respiratory cycle is smaller than a difference between the first respiratory cycle and the natural respiratory cycle.

### BRIEF DESCRIPTION OF THE DRAWINGS

An exemplary embodiment of the present disclosure will be described in detail based on the following figures, wherein:

FIG. 1 is a schematic diagram illustrating a configuration of a physical-mental conditioning apparatus according to the present exemplary embodiment;

FIG. 2 is a block diagram illustrating a configuration of the physical-mental conditioning apparatus according to the present exemplary embodiment;

FIG. 3 is a diagram illustrating an indicator of the degree of relaxation and that of the degree of physical-mental activity of a user according to the present exemplary embodiment;

FIG. 4 is a block diagram illustrating a hardware configuration of a control device according to the present exemplary embodiment;

FIG. 5 is a block diagram illustrating functional configurations of the control device according to the present exemplary embodiment;

FIG. 6 is a graph illustrating an example relation between a respiratory cycle when breathing of a user is regulated with a presentation device according to the present exemplary embodiment and the degree of relaxation and the degree of physical-mental activity;

FIG. 7 is a flowchart illustrating a flow of a presentation control process that is performed by the control device according to the present exemplary embodiment;

FIG. 8 is a diagram illustrating a decrease state where the illuminance of an illumination device according to the present exemplary embodiment is decreased;

FIG. 9 is a diagram illustrating a flow of a process in a case where increasing of the degree of relaxation is to take precedence in the physical-mental conditioning apparatus according to the present exemplary embodiment;

FIG. 10 is a diagram illustrating a flow of a process in a case where giving of the feeling of awakening is to take precedence in the physical-mental conditioning apparatus according to the present exemplary embodiment;

FIG. 11 is a diagram illustrating an increase state where the illuminance of the illumination device according to the present exemplary embodiment is increased; and

FIG. 12 is a schematic diagram illustrating a configuration of a presentation device according to a modification.

### DETAILED DESCRIPTION

Hereinafter, an example of an exemplary embodiment of the present disclosure will be described with reference to the drawings.

### Physical-Mental Conditioning Apparatus 10

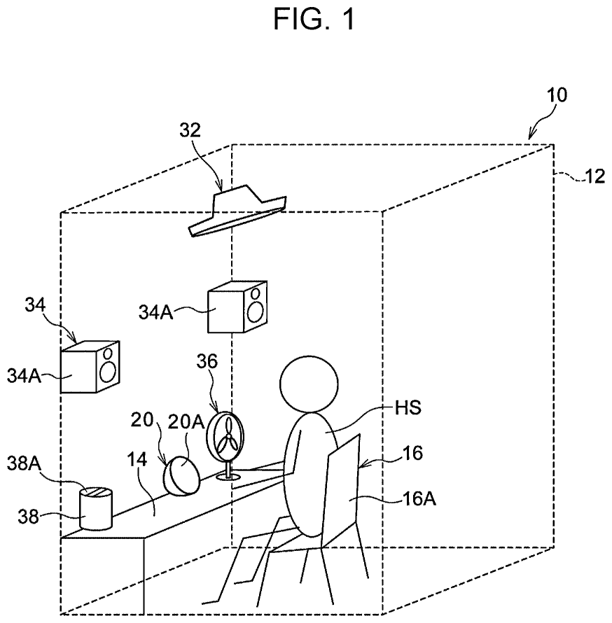

First, a configuration of a physical-mental conditioning apparatus 10 (hereinafter simply referred to as “conditioning apparatus 10”) according to the present exemplary embodiment will be described. FIG. 1 is a schematic diagram illustrating a configuration of the conditioning apparatus 10 according to the present exemplary embodiment.

The conditioning apparatus 10 illustrated in FIG. 1 is an apparatus for conditioning the mind and body of a person. Specifically, the conditioning apparatus 10 regulates breathing of a conditioning target person HS to thereby condition the mind and body of the conditioning target person HS. More specifically, the conditioning apparatus 10 regulates breathing of the conditioning target person HS and provides stimuli to the conditioning target person HS to thereby adjust the degree of relaxation and the degree of physical-mental activity of the conditioning target person HS. Further, the conditioning apparatus 10 adjusts the degree of relaxation and the degree of physical-mental activity of the conditioning target person HS as follows. The conditioning apparatus 10 increases the degree of relaxation of the conditioning target person HS to make the conditioning target person HS relaxed, or decreases, and thereafter, increases the degree of physical-mental activity of the conditioning target person HS to make the conditioning target person HS refreshed (that is, awakened).

The conditioning target person HS is a person whose mind and body are conditioned with the conditioning apparatus 10. In other words, the conditioning target person HS may be regarded as a user who uses the conditioning apparatus 10. Hereinafter, the conditioning target person HS may be referred to as “user HS”.

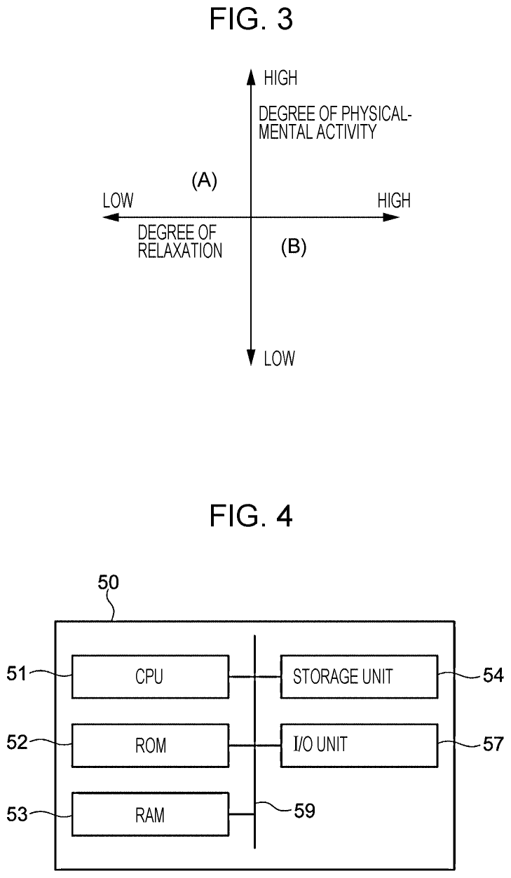

As illustrated in FIG. 3, the degree of relaxation (horizontal axis) is an indicator indicating whether the user HS is relaxed or the user HS is stressed. A high degree of relaxation indicates that the user HS is relaxed, and a low degree of relaxation indicates that the user HS is stressed. The degree of physical-mental activity (vertical axis) is an indicator indicating the vitality and the degree of awakening of the user HS. A high degree of physical-mental activity indicates that the user HS is awakened, is full of vitality, or feels active. A low degree of physical-mental activity indicates that the user HS is sleepy, has difficulty in paying attention or maintaining concentration, or becomes demotivated.

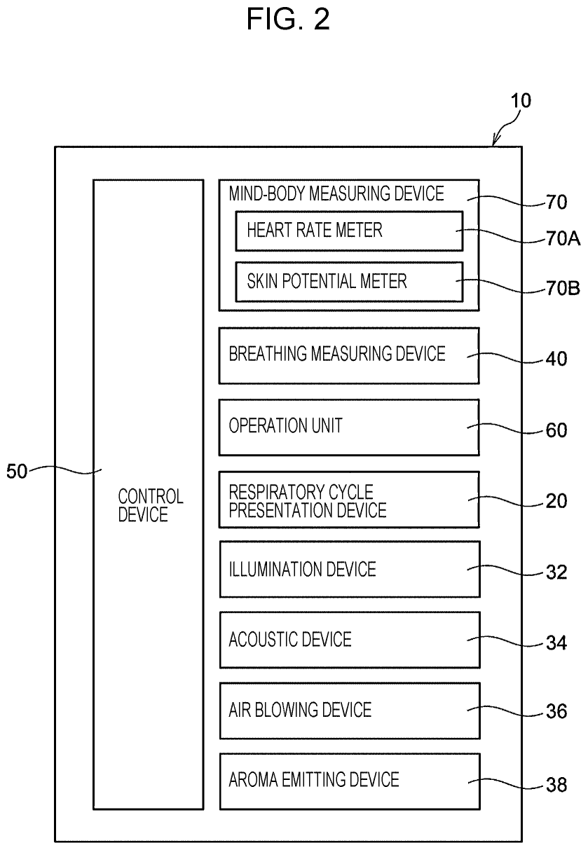

As illustrated in FIG. 1, specifically, the conditioning apparatus 10 includes a conditioning room 12, a table 14, and a chair 16. Further, as illustrated in FIG. 1 and FIG. 2, the conditioning apparatus 10 includes a respiratory cycle presentation device 20, an illumination device 32, an acoustic device 34, an air blowing device 36, and an aroma emitting device 38. Further, as illustrated in FIG. 2, the conditioning apparatus 10 includes a breathing measuring device 40, a mind-body measuring device 70, an operation unit 60, and a control device 50.

Specific configurations of the respective sections (the conditioning room 12, the table 14, the chair 16, the respiratory cycle presentation device 20, the illumination device 32, the acoustic device 34, the air blowing device 36, the aroma emitting device 38, the breathing measuring device 40, the mind-body measuring device 70, the operation unit 60, and the control device 50) of the conditioning apparatus 10 will be described below.

### Conditioning Room 12, Table 14, and Chair 16

The conditioning room 12 illustrated in FIG. 1 is a room (that is, a space) in which the mind and body of the user HS are conditioned. In the conditioning room 12, entry of outside light is limited. In the conditioning room 12, the table 14, the chair 16, the respiratory cycle presentation device 20, the illumination device 32, the acoustic device 34, the air blowing device 36, and the aroma emitting device 38 are provided, as illustrated in FIG. 1.

The chair 16 is an item for the user HS to be seated, as illustrated in FIG. 1. The chair 16 has a backrest 16A for the user HS to recline.

The table 14 functions as a surface on which the respiratory cycle presentation device 20, the air blowing device 36, and the aroma emitting device 38 are placed, as illustrated in FIG. 1.

### Respiratory Cycle Presentation Device 20

The respiratory cycle presentation device 20 (hereinafter simply referred to as “presentation device 20”) illustrated in FIG. 1 is a device that presents a respiratory cycle. Specifically, the presentation device 20 presents to the user HS a respiratory cycle through at least some of the five senses of the user HS. More specifically, the presentation device 20 presents to the user HS a respiratory cycle through the visual sense of the user HS. More specifically, the presentation device 20 is configured as follows.

The presentation device 20 has a display screen 20A on which a lamp blinks. The presentation device 20 is placed on the table 14 such that the display screen 20A faces the user HS seated on the chair 16. More specifically, the presentation device 20 is arranged on the table 14 such that the display screen 20A is located near the center of the sight of the user HS seated on the chair 16 in a natural position.

The presentation device 20 turns on the lamp at, for example, the start point of the respiratory cycle to present the respiratory cycle to the user HS. When the user HS makes their breathing synchronize so as to start breathing in at the time when the lamp is turned on, the breathing of the user HS is regulated. The respiratory cycle is a period from the start point at which the user HS starts breathing in to the next start point. For the presentation device 20, the user HS is an example of a presentation target person to which the respiratory cycle is presented.

As the method for presentation by the presentation device 20, a configuration may be employed in which the lamp in the ON state is turned off at the start point of the respiratory cycle. As the method for presentation by the presentation device 20, a configuration may be employed in which the lamp is turned on or turned off at the start point of the respiratory cycle (that is, at the time point when the user HS starts breathing in) and at the time point when the user HS starts breathing out. Further, a configuration may be employed in which the lamp is turned on with the highest brightness at the time point when the user HS stops breathing in in the respiratory cycle, the lamp is turned on with the lowest brightness or turned off at the time point when the user HS stops breathing out, and the brightness of the lamp is continuously changed in synchronization with the rhythm of the breathing.

Further, as the method for presentation by the presentation device 20, a presentation method may be employed in which, for example, an image of, for example, a rectangular shape displayed on the display screen 20A is deformed in synchronization with the rhythm with which the user HS breathes in or breathes out. In this case, for example, the image may be gradually enlarged in synchronization with the timing at which the user HS is to breathe in, and the image may be gradually reduced in synchronization with the timing at which the user HS is to breathe out.

### Illumination Device 32, Acoustic Device 34, Air Blowing Device 36, and Aroma Emitting Device 38

The illumination device 32, the acoustic device 34, the air blowing device 36, and the aroma emitting device 38 correspond to an example of a stimulus providing device that provides a stimulus. Specifically, the illumination device 32, the acoustic device 34, the air blowing device 36, and the aroma emitting device 38 each provide a stimulus to the user HS through at least some of the five senses.

More specifically, the illumination device 32 is a device that provides a visual stimulus to the user HS. Specifically, the illumination device 32 is provided in the upper part of the conditioning room 12 so as to be located above the user HS seated on the chair 16. Accordingly, the illumination device 32 illuminates the user HS seated on the chair 16. Further, the illuminance and the color of light of the illumination device 32 are adjustable. Specifically, for example, the illuminance of the illumination device 32 on the table 14 is set to 600 lx in the initial state, and the illumination device 32 is allowed to transition from the initial state to a decrease state where the illuminance is decreased to 300 lx. The color of light of the illumination device 32 is switchable between, for example, white and blue.

The acoustic device 34 is a device that provides an auditory stimulus to the user HS. The acoustic device 34 has a pair of speakers 34A arranged to the right and to the left of the user HS seated on the chair 16. Accordingly, the acoustic device 34 plays a sound to the user HS seated on the chair 16 through the pair of speakers 34A.

The air blowing device 36 is a device that provides a tactile stimulus to the user HS. Specifically, the air blowing device 36 is placed on the table 14 such that the air blowing direction extends toward the user HS seated on the chair 16. Accordingly, the air blowing device 36 blows air to the user HS seated on the chair 16.

The aroma emitting device 38 is a device that provides an olfactory stimulus to the user HS. The aroma emitting device 38 is placed on the table 14. On the upper part of the aroma emitting device 38, an outlet 38A through which aromatic air is emitted is provided. The aroma emitting device 38 emits aromatic air through the outlet 38A to thereby provide an aroma to the user HS seated on the chair 16.

For the illumination device 32, the acoustic device 34, the air blowing device 36, and the aroma emitting device 38, the user HS is an example of the presentation target person to which stimuli are provided.

### Breathing Measuring Device 40

The breathing measuring device 40 is a measuring device that measures an amount (parameter) indicating the natural respiratory cycle of the user HS. As the breathing measuring device 40, for example, a respiration sensor that is put on the belly of the user HS to measure the natural respiratory cycle of the user HS is used. The natural respiratory cycle is the respiratory cycle of the user HS when the user HS is at rest.

As the breathing measuring device 40, for example, a displacement sensor that is provided on the chair 16 may be used. The displacement sensor measures the amount of displacement of the chair 16 that is displaced by breathing of the user HS as an amount that indicates the natural respiratory cycle of the user HS. In this configuration, the natural respiratory cycle of the user HS is calculated from the amount of displacement of the chair 16.

As the breathing measuring device 40, a heart rate meter that measures the heartbeat of the user HS may be used. With the heart rate meter, the heart rate of the user HS is measured as an amount that indicates the natural respiratory cycle of the user HS. In this configuration, the natural respiratory cycle of the user HS is calculated from the heart rate of the user HS.

### Mind-Body Measuring Device 70

The mind-body measuring device 70 is a device that measures biological signals emitted from the interior of the body due to biological phenomena to obtain the degree of relaxation and the degree of physical-mental activity of the user HS. Specifically, the mind-body measuring device 70 has a heart rate meter 70A that measures the heartbeat of the user HS and a skin potential meter 70B that measures the skin potential level (SPL) of the user HS.

### Operation Unit 60

The operation unit 60 is an operation unit for performing an operation of giving an instruction for starting physical-mental conditioning by the conditioning apparatus 10. The operation unit 60 has, for example, a conditioning start button that is operated by the user HS in a case of giving an instruction for starting physical-mental conditioning by the conditioning apparatus 10. In the present exemplary embodiment, when the conditioning start button is operated by the user HS, physical-mental conditioning by the conditioning apparatus 10 is started.

### Control Device 50

The control device 50 is an example of a presentation control device. The control device 50 is a device that controls operations of the respiratory cycle presentation device 20, the illumination device 32, the acoustic device 34, the air blowing device 36, and the aroma emitting device 38. FIG. 4 is a block diagram illustrating a hardware configuration of the control device 50.

As illustrated in FIG. 4, the control device 50 has functions of a computer and has a central processing unit (CPU) 51, a read-only memory (ROM) 52, a random access memory (RAM) 53, a storage unit 54, and an input/output (I/O) unit 57. The units of the control device 50 are connected to one another via a bus 59 so as to enable mutual communication. The CPU 51 is an example of a processor.

The CPU 51 is a central processing unit, executes various programs including a presentation control program, and controls operations of the units. That is, the CPU 51 reads a program from the ROM 52 or the storage unit 54 and executes the program while using the RAM 53 as a work area. In accordance with the program recorded to the ROM 52 or the storage unit 54, the CPU 51 controls the sections (specifically, the presentation device 20, the illumination device 32, the acoustic device 34, the air blowing device 36, the aroma emitting device 38, the breathing measuring device 40, and the mind-body measuring device 70) of the conditioning apparatus 10 and performs various types of arithmetic processing.

The ROM 52 stores various programs and various types of data. The RAM 53 functions as a work area and temporarily stores a program or data. The storage unit 54 is formed of, for example, a hard disk drive (HDD) or a solid state drive (SSD) and stores various programs including an operating system and various types of data. The I/O unit 57 connects the CPU 51 with each section of the conditioning apparatus 10.

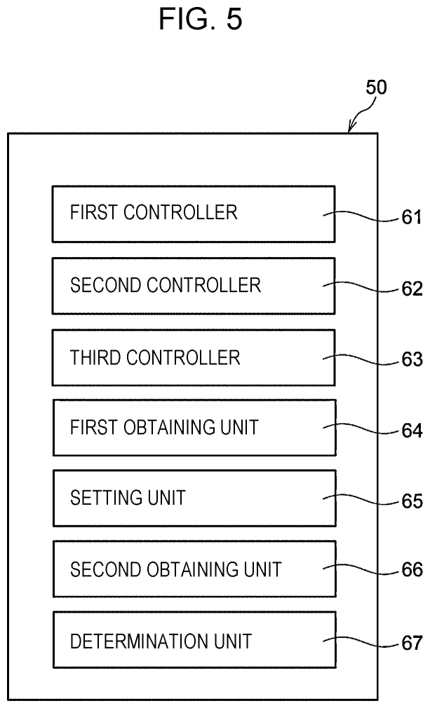

When executing the above-described programs, the control device 50 uses the above-described hardware resources to implement various functions. Functional configurations implemented by the control device 50 will be described. FIG. 5 is a block diagram illustrating example functional configurations of the control device 50.

As illustrated in FIG. 5, the control device 50 has a first controller 61, a second controller 62, a third controller 63, a first obtaining unit 64, a setting unit 65, a second obtaining unit 66, and a determination unit 67 as functional configurations. The functional configurations are implemented by the CPU 51 reading a control program stored in the ROM 52 or the storage unit 54 and executing the control program.

The first controller 61 performs for the presentation device 20 first control for presenting a first respiratory cycle longer than the natural respiratory cycle in a case of preferentially increasing the degree of relaxation. Specifically, the natural respiratory cycle is considered to take, for example, more than 3 seconds and 8 seconds or less. Specifically, the first respiratory cycle is set by the setting unit 65 so as to be within a range of, for example, more than 8 seconds and 15 seconds or less.

The second controller 62 performs for the presentation device 20 second control for presenting a second respiratory cycle in a case of preferentially giving the feeling of awakening, where the difference between the second respiratory cycle and the natural respiratory cycle is smaller than the difference between the first respiratory cycle and the natural respiratory cycle.

In the present exemplary embodiment, the degree of physical-mental activity is decreased, and thereafter, the degree of physical-mental activity is increased to give the feeling of awakening. Accordingly, the “case of preferentially giving the feeling of awakening” is an example of a “case of preferentially decreasing the degree of physical-mental activity”.

Examples of the “case of preferentially decreasing the degree of physical-mental activity” may include a “case where decreasing of the degree of physical-mental activity for making the user HS sleepy is to take precedence”. Examples of the “case of preferentially decreasing the degree of physical-mental activity” may include a “case where decreasing of the degree of physical-mental activity for subsequently putting the user HS into a physical-mental state other than the state where the user HS is sleepy is to take precedence”. That is, examples of the “case of preferentially decreasing the degree of physical-mental activity” may include a “case of decreasing the degree of physical-mental activity for a purpose other than the purpose of giving the feeling of awakening”.

Specifically, the second respiratory cycle is a cycle equal to or longer than the natural respiratory cycle and shorter than the first respiratory cycle. More specifically, the second respiratory cycle is the natural respiratory cycle. More specifically, the second respiratory cycle is set by the setting unit 65 so as to be within a range of, for example, more than 3 seconds and 8 seconds or less.

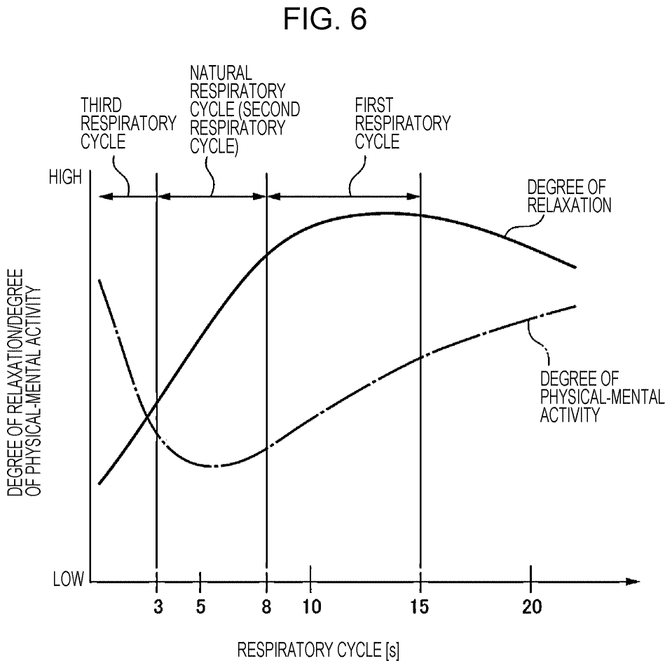

FIG. 6 is a graph illustrating an example relation between a respiratory cycle when breathing of the user HS is regulated with the presentation device 20 and the degree of relaxation and the degree of physical-mental activity.

As illustrated by the graph in FIG. 6, a respiratory cycle with which the degree of physical-mental activity is minimized and a respiratory cycle with which the degree of relaxation is maximized are different. Specifically, the degree of relaxation is maximized with a respiratory cycle (specifically, within a range of more than 8 seconds and 15 seconds or less) longer than the natural respiratory cycle. On the other hand, the degree of physical-mental activity is minimized with a respiratory cycle (specifically, within a range of more than 3 seconds and 8 seconds or less) equal to the natural respiratory cycle.

Accordingly, in the present exemplary embodiment, as the first respiratory cycle to be presented in a case of preferentially increasing the degree of relaxation, the respiratory cycle (specifically, within a range of more than 8 seconds and 15 seconds or less) with which the degree of relaxation is maximized is used. As the second respiratory cycle to be presented in a case of preferentially giving the feeling of awakening, the respiratory cycle (specifically, within a range of more than 3 seconds and 8 seconds or less) with which the degree of physical-mental activity is minimized is used.

Specifically, the second control performed by the second controller 62 is control for presenting the second respiratory cycle in the decrease state where presentation information (hereinafter referred to as “stimulus providing information”) presented by the illumination device 32 is decreased from that in the initial state.

Specifically, in the second control, the second respiratory cycle is presented in the decrease state where the illuminance of the illumination device 32 on the table 14 is decreased (for example, 300 lx) from that in the initial state where the illuminance is set to 600 lx. Further, in the second control, the second respiratory cycle is presented in a state where the light of the illumination device 32 is changed to blue from that in the initial state where the light of the illumination device 32 is white. Blue light has less wavelength components than white light has. When the light is changed to blue light, the stimulus providing information provided to the user HS is decreased.

In order to distance the user HS from surrounding noise so as not to be aware of the noise, in the second control, natural noise or white noise may be provided from the acoustic device 34.

The third controller 63 performs, for the illumination device 32 after performing the second control for the presentation device 20, third control for causing a transition to an increase state to occur where the stimulus providing information is increased from that in the decrease state.

Specifically, the third control causes a transition to the increase state to occur where the illuminance on the table 14 is increased (for example, 600 lx) from that in the decrease state where the illuminance is decreased to 300 lx. In the third control, the acoustic device 34, the air blowing device 36, and the aroma emitting device 38 are caused to provide stimuli to the user HS.

Further, in the third control, the presentation device 20 is caused to present a third respiratory cycle (see FIG. 6) shorter than the natural respiratory cycle. The third respiratory cycle is set within a range of, for example, 2 seconds or more and 3 seconds or less.

The first obtaining unit 64 obtains the result of measurement of the natural respiratory cycle measured by the breathing measuring device 40 prior to the first control and the second control. That is, the first obtaining unit 64 obtains information (hereinafter referred to as “cycle information”) about the natural respiratory cycle measured by the breathing measuring device 40 prior to the first control and the second control.

The setting unit 65 sets the first respiratory cycle and the second respiratory cycle on the basis of the result of measurement obtained by the first obtaining unit 64. That is, the setting unit 65 sets the first respiratory cycle and the second respiratory cycle on the basis of the cycle information obtained by the first obtaining unit 64.

The natural respiratory cycle differs among individuals. When the natural respiratory cycle differs, a respiratory cycle with which the degree of relaxation is maximized and a respiratory cycle with which the degree of physical-mental activity is minimized differ. Therefore, in the present exemplary embodiment, the first respiratory cycle is set with reference to a table of the first respiratory cycle that is associated in advance with the natural respiratory cycle. Further, the second respiratory cycle is set with reference to a table of the second respiratory cycle that is associated in advance with the natural respiratory cycle.

The second obtaining unit 66 obtains information (hereinafter referred to as “physical-mental information”) indicating the degree of relaxation and the degree of physical-mental activity of the user HS. Specifically, the second obtaining unit 66 obtains, as an example of the physical-mental information, information about the respiratory cycle, the heart rate, and the SPL of the user HS from the breathing measuring device 40, the heart rate meter 70A, and the skin potential meter 70B respectively.

The determination unit 67 determines, on the basis of the physical-mental information obtained by the second obtaining unit 66, which of the increasing of the degree of relaxation and the giving of the feeling of awakening is to take precedence. The “giving of the feeling of awakening” is an example of “decreasing of the degree of physical-mental activity”.

Specifically, on the basis of the physical-mental information obtained by the second obtaining unit 66, the determination unit 67 calculates the degree of relaxation and the degree of physical-mental activity and determines which of the increasing of the degree of relaxation and the giving of the feeling of awakening is to take precedence to increase a relatively low indicator among the degree of relaxation and the degree of physical-mental activity. That is, among the calculated degree of relaxation and the calculated degree of physical-mental activity, in a case where the degree of relaxation is relatively lower than the degree of physical-mental activity (corresponding to, for example, the region (A) in FIG. 3), the determination unit 67 determines that increasing of the degree of relaxation is to take precedence.

Further, among the calculated degree of relaxation and the calculated degree of physical-mental activity, in a case where the degree of physical-mental activity is relatively lower than the degree of relaxation (corresponding to, for example, the region (B) in FIG. 3), the determination unit 67 determines that giving of the feeling of awakening is to take precedence.

In a case where there is no relative difference between the calculated degree of relaxation and the calculated degree of physical-mental activity, the determination unit 67 determines that, for example, a predetermined one (for example, increasing of the degree of relaxation) among the increasing of the degree of relaxation and the giving of the feeling of awakening is to take precedence.

### Operations According to the Present Exemplary Embodiment

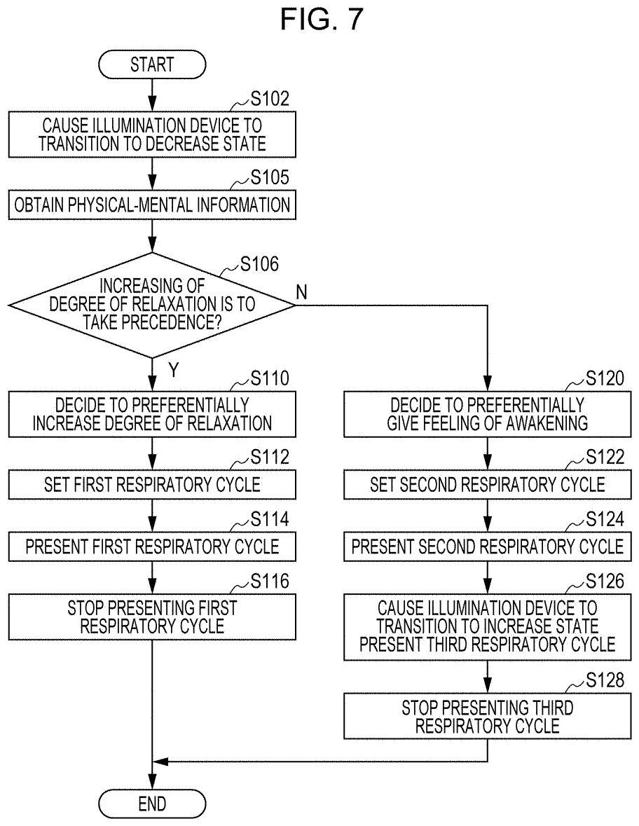

Next, operations in the present exemplary embodiment will be described. FIG. 7 is a flowchart illustrating a flow of a presentation control process that is performed by the control device 50.

The presentation control process is started when, for example, the user HS operates the conditioning start button of the operation unit 60. The user HS operates the conditioning start button of the operation unit 60 after putting on the breathing measuring device 40, which is a respiration sensor, the heart rate meter 70A, and the skin potential meter 70B. Before the start of the presentation control process, the illumination device 32 is in the initial state (see FIG. 1) where the illuminance on the table 14 is set to 600 lx.

The presentation control process according to the present exemplary embodiment is performed by the CPU 51 reading the presentation control program from the ROM 52 or the storage unit 54 and executes the presentation control program.

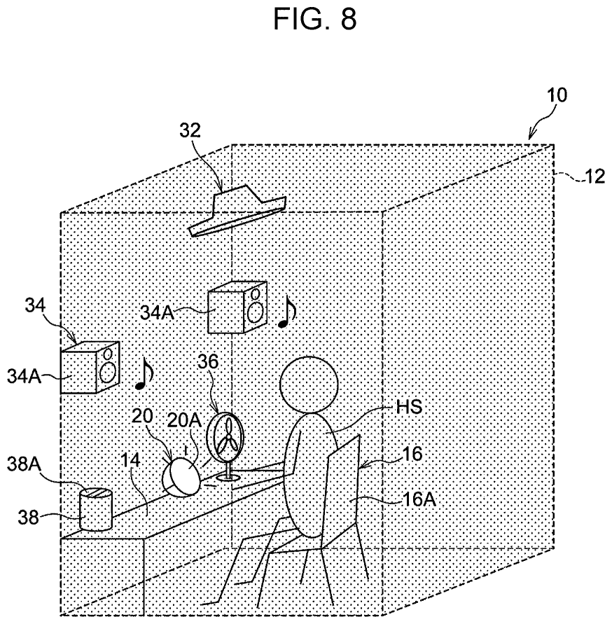

As illustrated in FIG. 7, when the presentation control process is started, the CPU 51 causes the illumination device 32 to transition from the initial state (see FIG. 1) to the decrease state (see FIG. 8) where the illuminance on the table 14 is decreased to 300 lx (step S102). At this time, the presentation device 20, the acoustic device 34, the air blowing device 36, and the aroma emitting device 38 remain stopped, and the user HS is put at rest.

Next, the CPU 51 causes the breathing measuring device 40, the heart rate meter 70A, and the skin potential meter 70B to measure the natural respiratory cycle, the heart rate, and the SPL of the user HS to obtain information about the natural respiratory cycle, the heart rate, and the SPL (step S105). The information about the natural respiratory cycle, the heart rate, and the SPL is an example of physical-mental information indicating the degree of relaxation and the degree of physical-mental activity of the user HS.

Next, the CPU 51 determines whether increasing of the degree of relaxation is to take precedence (step S106). In a case where the CPU 51 determines in step S106 that increasing of the degree of relaxation is to take precedence, the CPU 51 decides to preferentially increase the degree of relaxation (step S110), and the flow proceeds to step S112.

In a case where the CPU 51 determines in step S106 that increasing of the degree of relaxation is not to take precedence, the CPU 51 decides to preferentially give the feeling of awakening (step S120), and the flow proceeds to step S122.

In step S112, the CPU 51 sets the first respiratory cycle on the basis of the natural respiratory cycle. The CPU 51 sets the first respiratory cycle with reference to, for example, the table of the first respiratory cycle that is associated in advance with the natural respiratory cycle.

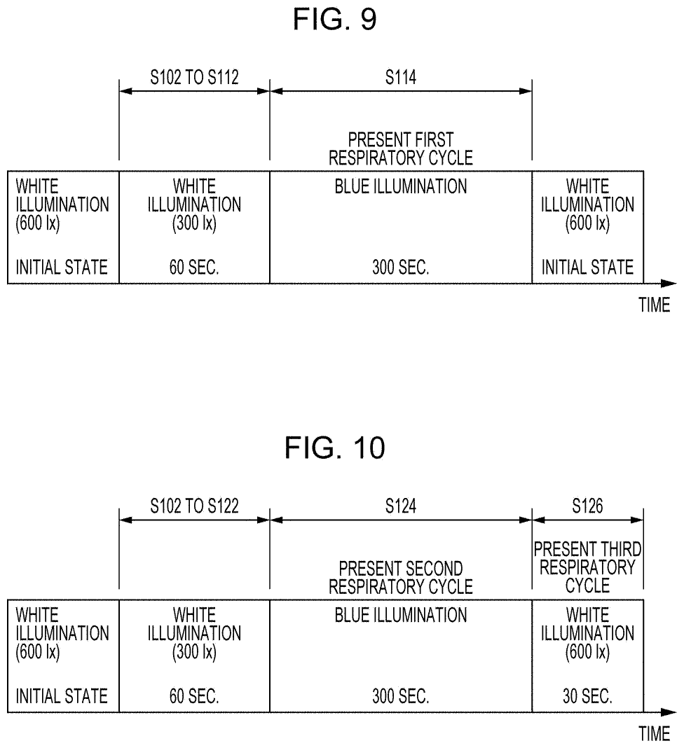

During the period from step S102 to step S112, the user HS is kept at rest. Further, as illustrated in FIG. 9, during the period, the illumination device 32 is kept in the decrease state where the illuminance on the table 14 is 300 lx. The period is set to, for example, 60 seconds.

Next, in step S114, the CPU 51 causes the presentation device 20 to present the first respiratory cycle (first control). Specifically, for example, the presentation device 20 turns on the lamp on the display screen 20A at the start point of the first respiratory cycle to present the first respiratory cycle to the user HS. When the user HS makes their breathing synchronize so as to start breathing in at the time when the lamp is turned on, the breathing of the user HS is regulated.

When the presentation device 20 presents the first respiratory cycle, the CPU 51 causes the illumination device 32 to change the color of its illumination to blue. Further, the CPU 51 causes the acoustic device 34 to output the natural sound of forests.

Next, the CPU 51 stops the presentation device 20 and the acoustic device 34 after the elapse of a predetermined period since the start of presentation of the first respiratory cycle by the presentation device 20 (step S116) and restores the illumination device 32 to the initial state, and the presentation control process ends. The predetermined period is set to, for example, 300 seconds (see FIG. 9).

In step S116, the CPU 51 stops the presentation device 20 and the acoustic device 34 after the elapse of the predetermined period; however, the presentation control process is not limited to this. For example, a configuration may be employed in which the CPU 51 detects an increase in the degree of relaxation of the user HS on the basis of information about the natural respiratory cycle, the heart rate, and the SPL obtained from the breathing measuring device 40, the heart rate meter 70A, and the skin potential meter 70B, and stops the presentation device 20 and the acoustic device 34 after the elapse of a predetermined period since the detection.

On the other hand, in step S122, the CPU 51 sets the second respiratory cycle on the basis of the natural respiratory cycle. The CPU 51 sets the second respiratory cycle with reference to, for example, the table of the second respiratory cycle that is associated in advance with the natural respiratory cycle.

During the period from step S102 to step S122, the user HS is kept at rest. Further, as illustrated in FIG. 10, during the period, the illumination device 32 is kept in the decrease state where the illuminance on the table 14 is 300 lx. The period is set to, for example, 60 seconds.

Next, in step S124, the CPU 51 causes the presentation device 20 to present the second respiratory cycle (second control). Specifically, for example, the presentation device 20 turns on the lamp on the display screen 20A at the start point of the second respiratory cycle to present the second respiratory cycle to the user HS. When the user HS makes their breathing synchronize so as to start breathing in at the time when the lamp is turned on, the breathing of the user HS is regulated.

When the presentation device 20 presents the second respiratory cycle, the CPU 51 causes the illumination device 32 to change the color of its illumination to blue. Further, the CPU 51 causes the acoustic device 34 to output the natural sound of forests.

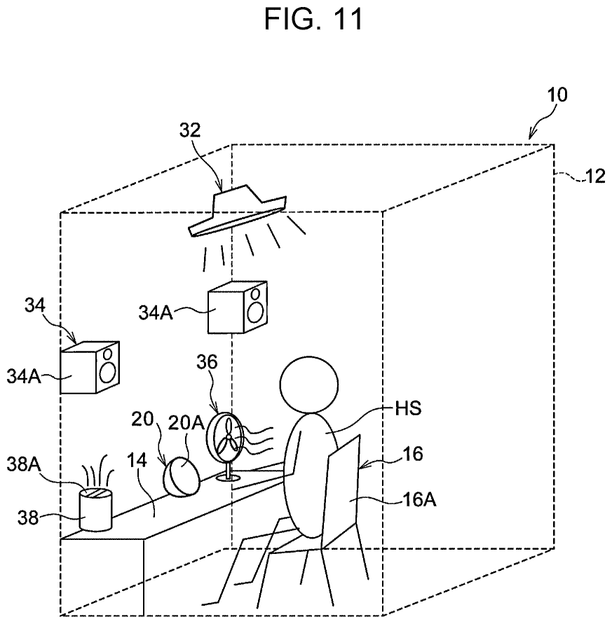

Next, in response to the elapse of a predetermined period since the start of presentation of the second respiratory cycle by the presentation device 20, in step S126, the CPU 51 causes the illumination device 32 to transition to the increase state where the illuminance of the illumination device 32 is increased to 600 lx (see FIG. 11) and causes the presentation device 20 to present the third respiratory cycle shorter than the natural respiratory cycle (third control). At this time, the CPU 51 stops the acoustic device 34 and causes the air blowing device 36 and the aroma emitting device 38 to start operating, as illustrated in FIG. 11. The predetermined period is set to, for example, 300 seconds (see FIG. 10).

As described above, the flow proceeds to step S126 after the elapse of the predetermined period; however, the presentation control process is not limited to this. For example, a configuration may be employed in which the CPU 51 detects a decrease in the degree of physical-mental activity of the user HS on the basis of information about the natural respiratory cycle, the heart rate, and the SPL obtained from the breathing measuring device 40, the heart rate meter 70A, and the skin potential meter 70B, and the flow proceeds to step S126 after the elapse of a predetermined period since the detection.

Next, the CPU 51 stops the presentation device 20, the air blowing device 36, and the aroma emitting device 38 after the elapse of a predetermined period since the start of presentation of the third respiratory cycle by the presentation device 20 (step S128), and the presentation control process ends. The predetermined period is set to, for example, 30 seconds (see FIG. 10).

In step S128, the CPU 51 stops the presentation device. 20, the air blowing device 36, and the aroma emitting device 38 after the elapse of the predetermined period; however, the presentation control process is not limited to this. For example, a configuration may be employed in which the CPU 51 detects an increase in the degree of physical-mental activity (that is, the feeling of awakening has been given) on the basis of information about the natural respiratory cycle, the heart rate, and the SPL obtained from the breathing measuring device 40, the heart rate meter 70A, and the skin potential meter 70B, and stops the presentation device 20, the air blowing device 36, and the aroma emitting device 38 after the elapse of a predetermined period since the detection.

As described above, in the present exemplary embodiment, in a case where giving of the feeling of awakening is to take precedence, the CPU 51 causes the second respiratory cycle to be presented, where the difference between the second respiratory cycle and the natural respiratory cycle is smaller than the difference between the first respiratory cycle and the natural respiratory cycle.

Here, the user HS needs to concentrate their attention on the display screen 20A of the presentation device 20 in order to make their breathing synchronize with the respiratory cycle presented by the presentation device 20. With a configuration (hereinafter referred to as “first configuration”) in which the first respiratory cycle longer than the natural respiratory cycle is presented also in a case where giving of the feeling of awakening is to take precedence, the user HS increasingly concentrates their attention on the display screen 20A of the presentation device 20, which hinders the degree of physical-mental activity from decreasing. As a result, the user HS fails to obtain a sufficient feeling of awakening.

On the other hand, in the present exemplary embodiment, in the case where giving of the feeling of awakening is to take precedence, the CPU 51 causes the second respiratory cycle to be presented, where the difference between the second respiratory cycle and the natural respiratory cycle is smaller than the difference between the first respiratory cycle and the natural respiratory cycle. Therefore, unlike with the first configuration, the user HS naturally makes their breathing synchronize without concentrating their attention on the display screen 20A of the presentation device 20.

Accordingly, with the configuration of the present exemplary embodiment, unlike with the first configuration, the degree of physical-mental activity of the user HS is decreased. As a result, the user HS obtains a sufficient feeling of awakening.

Further, in the present exemplary embodiment, specifically, the second respiratory cycle is a cycle equal to or longer than the natural respiratory cycle and shorter than the first respiratory cycle. With a configuration (hereinafter referred to as “second configuration”) in which the second respiratory cycle is a cycle shorter than the natural respiratory cycle, the degree of physical-mental activity is more likely to increase than in a case where the second respiratory cycle is longer than the natural respiratory cycle (see FIG. 6).

On the other hand, in the present exemplary embodiment, the second respiratory cycle is a cycle equal to or longer than the natural respiratory cycle and shorter than the first respiratory cycle. Therefore, unlike with the second configuration, the degree of physical-mental activity of the user HS is decreased. As a result, the user HS obtains a sufficient feeling of awakening.

Further, specifically, in the present exemplary embodiment, the second respiratory cycle is the natural respiratory cycle. Therefore, unlike with a configuration (hereinafter referred to as “third configuration”) in which the second respiratory cycle is a cycle other than the natural respiratory cycle, the user HS naturally makes their breathing synchronize without concentrating their attention on the display screen 20A of the presentation device 20.

Accordingly, with the configuration of the present exemplary embodiment, unlike with the third configuration, the degree of physical-mental activity of the user HS is decreased. As a result, the user HS obtains a sufficient feeling of awakening.

In the present exemplary embodiment, as described above, the presentation device 20 presents, in the second control, the second respiratory cycle in the decrease state where the illuminance of the illumination device 32 on the table 14 is decreased (for example, 300 lx) from that in the initial state where the illuminance is set to 600 lx.

With a configuration (hereinafter referred to as “fourth configuration”) in which the presentation device 20 presents the second respiratory cycle while the illumination device 32 remains in the initial state, the degree of physical-mental activity is likely to increase due to a visual stimulus provided by the illumination device 32.

On the other hand, in the present exemplary embodiment, the presentation device 20 presents the second respiratory cycle while the illumination device 32 is in the decrease state. Therefore, unlike with the fourth configuration, the degree of physical-mental activity of the user HS is decreased. As a result, the user HS obtains a sufficient feeling of awakening.

In the present exemplary embodiment, as described above, the CPU 51 performs, for the illumination device 32 after performing the second control for the presentation device 20, the third control for causing a transition to the increase state to occur where the stimulus providing information is increased from that in the decrease state.

With a configuration (hereinafter referred to as “fifth configuration”) in which the decrease state where the presentation information is decreased is maintained after the second control has been performed for the respiratory cycle presentation device, the degree of physical-mental activity that has been decreased is less likely to increase. As a result, the user HS fails to obtain a sufficient feeling of awakening.

On the other hand, in the present exemplary embodiment, after the second control has been performed for the presentation device 20, a transition to the increase state is caused to occur where the stimulus providing information is increased from that in the decrease state. Therefore, unlike with the fifth configuration, the degree of physical-mental activity that has been decreased is likely to increase. As a result, the user HS obtains a sufficient feeling of awakening.

Further, in the present exemplary embodiment, the first respiratory cycle and the second respiratory cycle are set on the basis of information about the natural respiratory cycle measured by the breathing measuring device 40. Accordingly, unlike with a configuration (hereinafter referred to as “sixth configuration”) in which the first respiratory cycle and the second respiratory cycle are set by the user HS as desired, the first respiratory cycle and the second respiratory cycle become cycles that reflect the natural respiratory cycle. Therefore, the user HS naturally makes their breathing synchronize without concentrating their attention on the display screen 20A of the presentation device 20. Accordingly, with the configuration of the present exemplary embodiment, unlike with the sixth configuration, the degree of physical-mental activity of the user HS is decreased. As a result, the user HS obtains a sufficient feeling of awakening.

Further, in the present exemplary embodiment, the CPU 51 determines, on the basis of the obtained physical-mental information, which of the increasing of the degree of relaxation and the giving of the feeling of awakening is to take precedence. Accordingly, unlike with a configuration in which the user HS determines as desired which of the increasing of the degree of relaxation and the obtaining of the feeling of awakening is to take precedence, it is determined, in accordance with the objective physical-mental state of the user HS, which of the increasing of the degree of relaxation and the giving of the feeling of awakening is to take precedence. Therefore, variations in the effect of conditioning of the physical-mental state of the user HS are suppressed.

### Modification of Presentation Device 20

In the present exemplary embodiment, the presentation device 20 is used as an example of the respiratory cycle presentation device; however, the present exemplary embodiment is not limited to this. For example, as an example of the respiratory cycle presentation device, a presentation device that presents the respiratory cycle to the user HS through some of the five senses (the auditory sense, tactile sense, and so on) except the visual sense may be used. Examples of a configuration for presenting the respiratory cycle through the auditory sense include a configuration in which a sound is generated at the start point of the respiratory cycle. Examples of a configuration for presenting the respiratory cycle through the tactile sense include a configuration in which a stimulus is provided to the skin at the start point of the respiratory cycle.

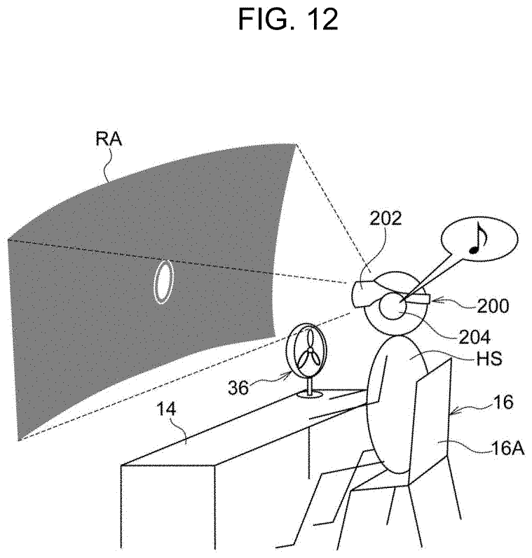

Further, as an example of the respiratory cycle presentation device, a virtual reality (VR) headset 200 may be used, as illustrated in FIG. 12. The VR headset 200 has a display device 202 that is put on the head and headphones 204. With the VR headset 200, the respiratory cycle is presented to the user HS by the display device 202 through the visual sense and by the headphones 204 through the auditory sense. The reference characters RA in FIG. 12 indicates a video image visually recognized by the user HS with the display device 202.

Further, as an example of the respiratory cycle presentation device, a portable terminal, such as a smartphone or a tablet computer, owned by the user HS may be used as the presentation device 20.

### Other Modifications

In the present exemplary embodiment, the second respiratory cycle is the natural respiratory cycle; however, the present exemplary embodiment is not limited to this. For example, the second respiratory cycle may be a cycle that exceeds the natural respiratory cycle and that is shorter than the first respiratory cycle. The second respiratory cycle may be a cycle shorter than the natural respiratory cycle as long as the difference between the second respiratory cycle and the natural respiratory cycle is smaller than the difference between the first respiratory cycle and the natural respiratory cycle.

In the present exemplary embodiment, information about the natural respiratory cycle measured by the breathing measuring device 40 is obtained by the first obtaining unit 64, and the first respiratory cycle and the second respiratory cycle are set on the basis of the information about the natural respiratory cycle; however, the present exemplary embodiment is not limited to this. For example, information about the natural respiratory cycle input by the user HS through the operation unit 60 or the like may be obtained by the first obtaining unit 64, and the first respiratory cycle and the second respiratory cycle may be set on the basis of the information about the natural respiratory cycle. In this case, for example, the user HS may measure in advance their natural respiratory cycle by themselves and input the natural respiratory cycle through the operation unit 60 or the like.

In the present exemplary embodiment, the determination unit 67 determines, on the basis of physical-mental information (for example, information about the respiratory cycle, the heart rate, and the SPL) obtained by the second obtaining unit 66, which of the increasing of the degree of relaxation and the giving of the feeling of awakening is to take precedence; however, the present exemplary embodiment is not limited to this. For example, a configuration may be employed in which the user HS selects whether increasing of the degree of relaxation is to take precedence or obtaining of the feeling of awakening is to take precedence. In this configuration, for example, the user HS performs a selection operation through the operation unit 60.

Further, in the present exemplary embodiment, information about the respiratory cycle, the heart rate, and the SPL of the user HS measured by the breathing measuring device 40, the heart rate meter 70A, and the skin potential meter 70B is obtained by the second obtaining unit 66; however, the present exemplary embodiment is not limited to this. For example, a configuration may be employed in which the second obtaining unit 66 obtains information not from measurement by the measuring instruments but from a questionnaire or the like and it is determined, on the basis of the information, which of the increasing of the degree of relaxation and the giving of the feeling of awakening is to take precedence.

The present disclosure is not limited to the above-described exemplary embodiment, and various modifications, changes, and improvements may be made without departing from the spirit thereof. For example, plural modifications among the above-described modifications may be combined as appropriate to make a configuration.

In the embodiment above, the term “processor” refers to hardware in a broad sense. Examples of the processor include general processors (e.g., the CPU described above) and dedicated processors (e.g., GPU: Graphics Processing Unit, ASIC: Application-Specific Integrated Circuit, FPGA: Field Programmable Gate Array, and a programmable logic device).

In the embodiment above, the term “processor” is broad enough to encompass one processor or plural processors in collaboration which are located physically apart from each other but may work cooperatively. The order of operations of the processor is not limited to one described in the embodiment above, and may be changed.

### Evaluation

The physical-mental state of the user HS is evaluated for a first experiment example (an awakening mode), a second experiment example (a relaxation mode), and a comparative example.

In this evaluation, the Roken Arousal Scale (RAS) is used as a subjective evaluation indicator to indicate the degree of physical-mental activity and the degree of relaxation of the user HS as scores and obtain the amount of change in each score before and after the use of the device. Further, from information about variations in heartbeat obtained by the heart rate meter 70A, the coefficient of variation of R-R interval (CVRR) that indicates the degree of activity of the entire autonomic nerve and the coefficient of component variance of high frequency (ccvHF) that indicates the degree of activity of the parasympathetic nerve and that is considered to reflect the relaxation state are calculated to obtain the amount of change before and after the use of the device.

### First Experiment Example (Awakening Mode)

In the first experiment example, step S102 to S106 and step S120 to S128 in the presentation control process described above are performed for 19 test subjects.

As a result, the score indicating the subjective degree of activity increases by an average of 2.3 points. The degree of activity of the autonomic nerve (CVRR) increases by an average of 55%.

### Comparative Example

In the comparative example, step S102 to S106 and step S120 to S128 in the presentation control process described above are performed for 10 test subjects under the conditions the same as those in the first experiment example except that the second respiratory cycle is fixed to 10 seconds. The second respiratory cycle of 10 seconds is a respiratory cycle longer than any values of the second respiratory cycle that are set in the first experiment example.

As a result, the amount of change in the score indicating the subjective degree of activity is an average of 0.3 points, and the subjective degree of activity decreases after the end of the procedure in two test subjects. The degree of activity of the autonomic nerve (CVRR) increases by an average of 8%. Accordingly, it is found that the feeling of awakening is obtained to a higher degree in the first experiment example than in the comparative example.

### Second Experiment Example (Relaxation Mode)

In the second experiment example, step S102 to S106 and step S110 to S116 in the presentation control process described above are performed for 25 test subjects.

As a result, the score indicating the subjective degree of relaxation increases by an average of 2.2 points. Further, the degree of activity of the parasympathetic nerve (ccvHF) increases by an average of 28%.

The foregoing description of the exemplary embodiment of the present disclosure has been provided for the purposes of illustration and description. It is not intended to be exhaustive or to limit the disclosure to the precise forms disclosed. Obviously, many modifications and variations will be apparent to practitioners skilled in the art. The embodiment was chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. A presentation control device comprising
a processor configured to
perform first control for presenting a first respiratory cycle longer than a natural respiratory cycle for a respiratory cycle presentation device in a case of preferentially increasing a degree of relaxation, the respiratory cycle presentation device being a device that presents a respiratory cycle, and
perform second control for presenting a second respiratory cycle for the respiratory cycle presentation device in a case of preferentially decreasing a degree of physical-mental activity, the second respiratory cycle being a cycle for which a difference between the second respiratory cycle and the natural respiratory cycle is smaller than a difference between the first respiratory cycle and the natural respiratory cycle.

2. The presentation control device according to claim 1, wherein
the second respiratory cycle is a cycle equal to or longer than the natural respiratory cycle and shorter than the first respiratory cycle.

3. The presentation control device according to claim 2, wherein
the second respiratory cycle is the natural respiratory cycle.

4. The presentation control device according to claim 1, wherein
the second control is control for presenting the second respiratory cycle in a decrease state where presentation information presented by a stimulus providing device that provides a stimulus through at least some of five senses is decreased from the presentation information presented in an initial state.

5. The presentation control device according to claim 2, wherein
the second control is control for presenting the second respiratory cycle in a decrease state where presentation information presented by a stimulus providing device that provides a stimulus through at least some of five senses is decreased from the presentation information presented in an initial state.

6. The presentation control device according to claim 3, wherein
the second control is control for presenting the second respiratory cycle in a decrease state where presentation information presented by a stimulus providing device that provides a stimulus through at least some of five senses is decreased from the presentation information presented in an initial state.

7. The presentation control device according to claim 4, wherein
the processor is configured to further
perform, for the stimulus providing device after performing the second control for the respiratory cycle presentation device, third control for causing a transition to an increase state to occur where the presentation information is increased from the presentation information presented in the decrease state.

8. The presentation control device according to claim 5, wherein
the processor is configured to further
perform, for the stimulus providing device after performing the second control for the respiratory cycle presentation device, third control for causing a transition to an increase state to occur where the presentation information is increased from the presentation information presented in the decrease state.

9. The presentation control device according to claim 6, wherein
the processor is configured to further
perform, for the stimulus providing device after performing the second control for the respiratory cycle presentation device, third control for causing a transition to an increase state to occur where the presentation information is increased from the presentation information presented in the decrease state.

10. The presentation control device according to claim 1, wherein
the processor is configured to further
obtain, prior to the first control and the second control, a result of measurement by a measuring device that measures a natural respiratory cycle of a presentation target person, and
set the first respiratory cycle and the second respiratory cycle on the basis of the obtained result of measurement.

11. The presentation control device according to claim 2, wherein
the processor is configured to further
obtain, prior to the first control and the second control, a result of measurement by a measuring device that measures a natural respiratory cycle of a presentation target person, and
set the first respiratory cycle and the second respiratory cycle on the basis of the obtained result of measurement.

12. The presentation control device according to claim 3, wherein
the processor is configured to further
obtain, prior to the first control and the second control, a result of measurement by a measuring device that measures a natural respiratory cycle of a presentation target person, and
set the first respiratory cycle and the second respiratory cycle on the basis of the obtained result of measurement.

13. The presentation control device according to claim 4, wherein
the processor is configured to further
obtain, prior to the first control and the second control, a result of measurement by a measuring device that measures a natural respiratory cycle of a presentation target person, and
set the first respiratory cycle and the second respiratory cycle on the basis of the obtained result of measurement.

14. The presentation control device according to claim 5, wherein
the processor is configured to further
obtain, prior to the first control and the second control, a result of measurement by a measuring device that measures a natural respiratory cycle of a presentation target person, and
set the first respiratory cycle and the second respiratory cycle on the basis of the obtained result of measurement.

15. The presentation control device according to claim 1, wherein
the processor is configured to further
obtain information indicating a degree of relaxation and a degree of physical-mental activity of a presentation target person, and
determine, on the basis of the information, which of increasing of the degree of relaxation and decreasing of the degree of physical-mental activity is to take precedence.

16. The presentation control device according to claim 2, wherein
the processor is configured to further
obtain information indicating a degree of relaxation and a degree of physical-mental activity of a presentation target person, and
determine, on the basis of the information, which of increasing of the degree of relaxation and decreasing of the degree of physical-mental activity is to take precedence.

17. The presentation control device according to claim 3, wherein
the processor is configured to further
obtain information indicating a degree of relaxation and a degree of physical-mental activity of a presentation target person, and
determine, on the basis of the information, which of increasing of the degree of relaxation and decreasing of the degree of physical-mental activity is to take precedence.

18. The presentation control device according to claim 4, wherein
the processor is configured to further
obtain information indicating a degree of relaxation and a degree of physical-mental activity of a presentation target person, and
determine, on the basis of the information, which of increasing of the degree of relaxation and decreasing of the degree of physical-mental activity is to take precedence.

19. A non-transitory computer readable medium storing a presentation control program causing a computer to execute a process comprising:
performing first control for presenting a first respiratory cycle longer than a natural respiratory cycle for a respiratory cycle presentation device in a case of preferentially increasing a degree of relaxation, the respiratory cycle presentation device being a device that presents a respiratory cycle; and
performing second control for presenting a second respiratory cycle for the respiratory cycle presentation device in a case of preferentially decreasing a degree of physical-mental activity, the second respiratory cycle being a cycle for which a difference between the second respiratory cycle and the natural respiratory cycle is smaller than a difference between the first respiratory cycle and the natural respiratory cycle.

20. A respiratory cycle presentation device that
presents a first respiratory cycle longer than a natural respiratory cycle in a case of preferentially increasing a degree of relaxation, and
presents a second respiratory cycle in a case of preferentially decreasing a degree of physical-mental activity, the second respiratory cycle being a cycle for which a difference between the second respiratory cycle and the natural respiratory cycle is smaller than a difference between the first respiratory cycle and the natural respiratory cycle.
